(19) United States
(12) Patent Application Publication
(10) Pub. No.: US 2009/0099466 A1
(43) Pub. Date: Apr. 16, 2009

(54) METHOD AND SYSTEM FOR AUTOMATIC CUFF TYPE DETERMINATION

(75) Inventors: Jose N. Wong, Tampa, FL (US); Richard Medero, Tampa, FL (US); Grady W. Argo, Mulberry, FL (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/873,215

(22) Filed: Oct. 16, 2007

(51) Int. Cl. A61B 5/02

(52) U.S. Cl. 600/495; 600/490; 600/494; 600/499

(57) ABSTRACT

A method for identifying a non-invasive blood pressure cuff type is disclosed herein. The method includes inflating a cuff and obtaining a first pressure measurement in a non-invasive blood pressure system while inflating the cuff. The method also includes obtaining a second pressure measurement in the non-invasive blood pressure system while inflating the cuff and identifying a cuff type based on the first pressure measurement and the second pressure measurement. A corresponding blood pressure monitoring system is also provided.

### BACKGROUND OF THE INVENTION

The human heart muscle periodically contracts, forcing blood through the arteries. As a result of this pumping action, pressure pulses exist in these arteries and cause them to cyclically change volume. The minimum pressure for these pulses during a cardiac cycle is known as a diastolic pressure and the peak pressure during a cardiac cycle is known as a systolic pressure. A further pressure value, known as a “mean arterial pressure” (MAP), represents the time-weighted average of the blood pressure. The systolic pressure, MAP and diastolic pressure for a patient are useful in monitoring the cardiovascular state of the patient, and in treating disease.

A conventional method of measuring blood pressure is referred to as oscillometry. Typically, the measurement of blood pressure by oscillometry requires the inflation of a cuff to a pressure level above the patient's systolic pressure to fully occlude the artery. The blood pressure is then determined by measuring an oscillation amplitude at multiple cuff pressure levels during the deflation of the cuff.

In order for oscillometry to provide an accurate estimation of the patient's blood pressure, it is necessary that the cuff be of an appropriate type for the patient whose blood pressure is being estimated. For example a neonatal cuff is typically smaller in size than an adult cuff, and the neonatal cuff may differ from the adult cuff in other design parameters as well. Due to the differences between cuff types, it is common for a non-invasive blood pressure (NIBP) system to have two or more algorithms in order to accommodate the range of cuff types available. For the purposes of this disclosure, the algorithm is defined to include control of the inflation of the cuff, control of the deflation of the cuff, and the calculation of the patient's blood pressure parameters.

In conventional systems, a clinician manually inputs the cuff type being used and this input determines the algorithm used to estimate the patient's blood pressure. The problem is that if the clinician inputs the wrong cuff type, an incorrect algorithm will be used which could result in patient discomfort. For example, if an adult algorithm is used with a neonatal cuff on an infant, the adult algorithm could result in the inflation of the cuff to a pressure higher than what is comfortable for the infant. Additionally, using the wrong algorithm for a particular cuff type may result in a less accurate blood pressure estimation.

### BRIEF DESCRIPTION OF THE INVENTION

The above-mentioned shortcomings, disadvantages and problems are addressed herein which will be understood by reading and understanding the following specification.

In an embodiment, a method for identifying a non-invasive blood pressure cuff type includes inflating a cuff, and obtaining a first pressure measurement and a second pressure measurement in the non-invasive blood pressure system while inflating the cuff. The method also includes identifying a cuff type based on the first pressure measurement and the second pressure measurement.

In an embodiment, a method for identifying a non-invasive blood pressure cuff type includes inflating a cuff and obtaining a first plurality of pressure measurements at a first location upstream relative to the cuff while inflating the cuff. The method also includes obtaining a second plurality of pressure measurements at a second location downstream relative to the cuff while inflating the cuff. The method also includes identifying a cuff type based on the first plurality of pressure measurements and the second plurality of pressure measurements.

In an embodiment, a blood pressure monitoring system includes a cuff and a first section of hose attached to the cuff. The blood pressure monitoring system also includes a first transducer operatively connected to the first section of hose. The first transducer is configured to obtain a first pressure measurement. The blood pressure monitoring system also includes a second section of hose attached to the cuff and a second transducer operatively connected to the second section of hose. The second transducer is configured to obtain a second pressure measurement. The blood pressure monitoring system also includes a controller operatively connected to the first transducer and the second transducer. The controller is configured to identify a cuff type based on the first pressure measurement and the second pressure measurement.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram illustrating a non-invasive blood pressure system in accordance with an embodiment;

FIG. 2 is a schematic diagram illustrating an adult non-invasive blood pressure system in accordance with an embodiment;

FIG. 3 is a schematic diagram illustrating a neonatal non-invasive blood pressure system in accordance with an embodiment;

FIG. 4 is a flow chart illustrating a method of determining cuff type in accordance with an embodiment; and

FIG. 5 is a graph of pressure differential versus time illustrating exemplary neonatal cuff data and exemplary adult cuff data.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

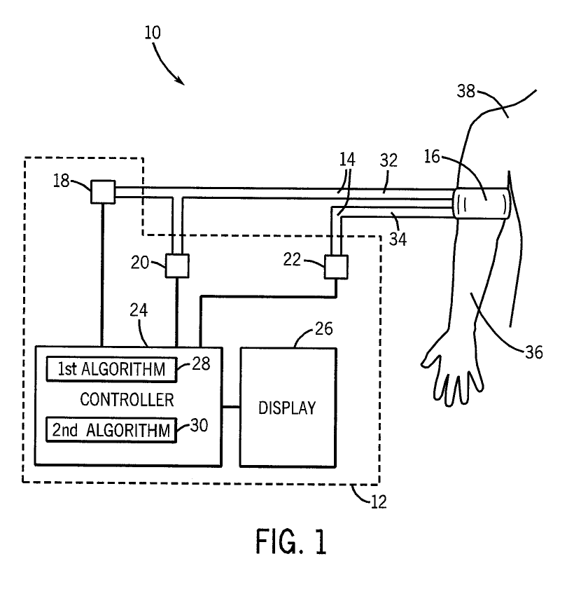

Referring to FIG. 1, a schematic representation of a non-invasive blood pressure (NIBP) system 10 is shown in accordance with an embodiment. The NIBP system 10 comprises a monitor 12, a hose system 14 and a cuff 16.

The monitor 12 comprises a generally box-shaped plastic housing (not shown) adapted to retain: a source of pressurized gas 18; a first transducer 20; a second transducer 22; a controller 24; and a display 26. The controller 24 is electronically attached to the source of pressurized gas 18, the first transducer 20, the second transducer 22, and the display 26. The source of pressurized gas 18, the first transducer 20, and the second transducer 22 are all pneumatically connected to the hose system 14, as will be described in detail hereinafter. The controller 24 comprises a first algorithm 28 and a second algorithm 30, which will both be described in detail hereinafter. The source of pressurized gas 18 may include a check valve (not shown) biased so as to allow pressurized gas to only flow in the direction away from the source of pressurized gas 18.

The hose system 14 is connected to the monitor 12 and to the cuff 16. The hose system 14 comprises a first section of hose 32 and a second section of hose 34. The first section of hose 32 and the second section of hose 34 pneumatically connect the monitor 12 to the cuff 16. The first section of hose 32 is pneumatically connected to the source of pressurized gas 18, the first transducer 20, and the cuff 16. The second section of hose 34 is pneumatically connected to the cuff 16 and the second transducer 22. The first section of hose 32 is located upstream from the second section of hose 34. For the purposes of this disclosure, the term “upstream” will be defined to include the direction towards the source of pressurized gas 18 and the term “downstream” will be defined to include the direction away from the source of pressurized gas 18. According to an embodiment, the first section of hose 32 and the second section of hose 34 may be generally parallel to each other.

The cuff 16 comprises one or more inflatable bladders (not shown) that can be selectively filled with gas from the source of pressurized gas 18. Although the cuff 16 is depicted around an arm 36 of a patient 38, it should be appreciated that the cuff 16 could also be disposed around a leg (not shown) or other limb (not shown). The cuff 16 is attached at the downstream end of the first section of hose 32 and to the upstream end of the second section of hose 34.

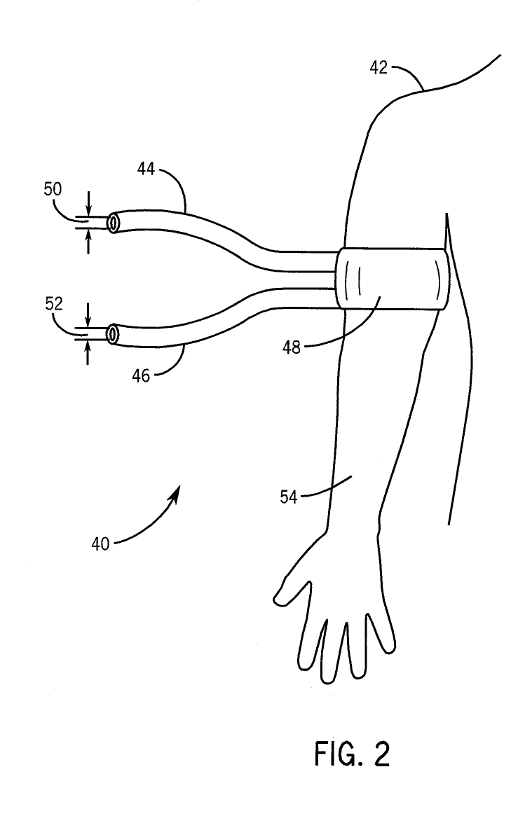

Referring to FIG. 2, a schematic representation of an adult NIBP system 40 is shown in accordance with an embodiment. The adult NIBP system 40 is attached to an adult patient 42. The adult NIBP system 40 comprises a first section of hose 44, a second section of hose 46, and an adult cuff 48. The first section of hose 44 defines a first internal diameter 50 and the second section of hose 46 defines a second internal diameter 52. The first section of hose 44 and the second section of hose 46 are connected to the adult cuff 48 which is schematically shown around an adult arm 54 of the adult patient 42.

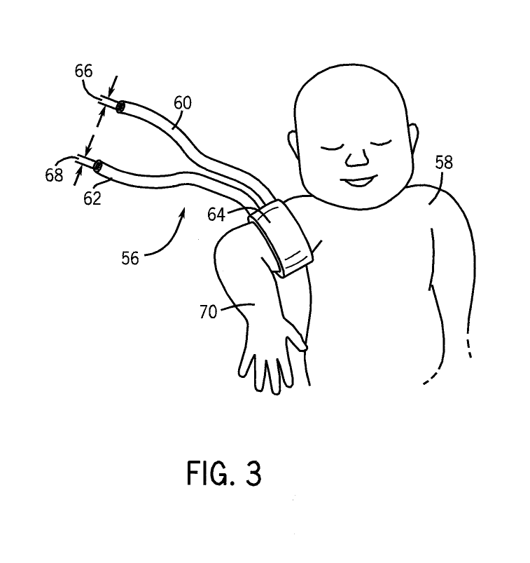

Referring to FIG. 3, a schematic representation of a neonatal NIBP system 56 is shown in accordance with an embodiment. The neonatal NIBP system 56 is attached to an infant patient 58. The neonatal NIBP system 56 comprises a first section of hose 60, a second section of hose 62, and a neonatal cuff 64. The first section of hose 60 defines a first internal diameter 66 and the second section of hose 62 defines a second internal diameter 68. The first section of hose 60 and the second section of hose 62 are connected to the neonatal cuff 64 which is schematically shown around an infant arm 70 of the infant patient 58.

Referring now to both FIG. 2 and FIG. 3, the first internal diameter 50 and the second internal diameter 52 of the adult NIBP system 40 are larger than the first internal diameter 66 and the second internal diameter 68 of the neonatal NIBP system 56. Because the first internal diameter 66 and the second internal diameter 68 are smaller than the first internal diameter 50 and the second internal diameter 52, they present a greater resistance to the flow of gas from a source of pressurized gas (not shown). By measuring and/or calculating a variable correlated with the resistance to the flow of gas in an NIBP system (not shown), it may be possible to identify the cuff type of the NIBP system.

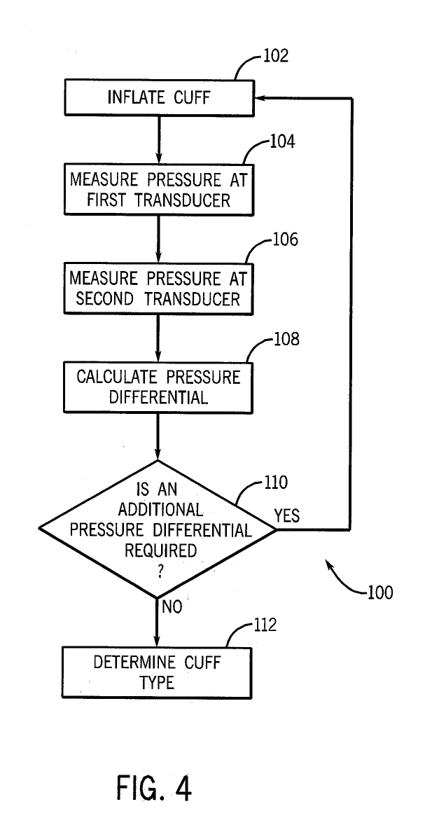

Having described the structure of the NIBP systems 40 and 56, a method 100 will be described hereinafter. FIG. 4 is a flow chart illustrating the method 100 in accordance with an embodiment. The individual blocks 102-112 of the flow chart represent steps that may be performed in accordance with the method 100. The technical effect of the method 100 is to determine a cuff type of an NIBP system 10 (shown in FIG. 1). The steps 102-112 of the method 100 need not be performed in the order shown.

Referring to FIGS. 1 and 4, at step 102, the cuff 16 is inflated. As part of step 102, the controller 24 communicates with the source of pressurized gas 18, causing gas to travel from the source of pressurized gas 18, through the first section of hose 32 and into the cuff 16. At step 104, the first transducer 20 obtains a pressure measurement within the first section of hose 32.

At step 106, the second transducer 22 obtains a pressure measurement within the second section of hose 34. According to one embodiment, the pressure measurements obtained during steps 104 and 106 are obtained generally simultaneously. The pressure measurements obtained during steps 104 and 106 are transmitted to the controller 24 prior to step 108. At step 108, a pressure differential is calculated by the controller 24. For the purposes of this disclosure, the pressure differential is defined to include a difference between two pressure measurements. For this disclosure, it should be understood that the implementation of the pressure differential could be replaced by the implementation of alternative methods of comparing pressure measurements. One example of an alternative method of comparing pressure measurements would be calculating a ratio of the pressure measurements obtained at the first transducer 20 and the second transducer 22. In the embodiment schematically represented by method 100, the pressure differential comprises the difference between the pressure measurement obtained at the first transducer 20 at step 104 and the pressure measurement obtained at the second transducer 22 at step 106.

At step 110, the controller 24 determines if an additional pressure differential calculation is required to identify cuff type. The controller 24 may make this determination by comparing the number of pressure differentials calculated to a previously determined target number of pressure differentials, or the controller may check to see if the cuff type may be determined based on the pressure differential(s) that have already been collected. If an additional pressure differential is required, the method 100 returns to step 102, where the source of pressurized gas 18 inflates the cuff 16 with additional gas. Steps 102-110 may be iterated as many times as necessary. It should be appreciated that iterations of steps 102-110 could be implemented so the pressure within the cuff 16 is increased in either a continuous or in a stepwise fashion. If an additional pressure differential is not required at step 110, the method 100 proceeds to step 112.

According to an alternate embodiment, steps 102-110 of the method 100 may be replaced by a generally equivalent sequence of steps wherein a pressure differential similar to that of step 108 is calculated based on a first average pressure measurement and a second average pressure measurement. The first average pressure measurement may, for example, comprise an average of two or more pressure measurements iteratively acquired from the first transducer 20. Similarly, the second average pressure measurement may comprise an average of two or more pressure measurements iteratively acquired from the second transducer 22.

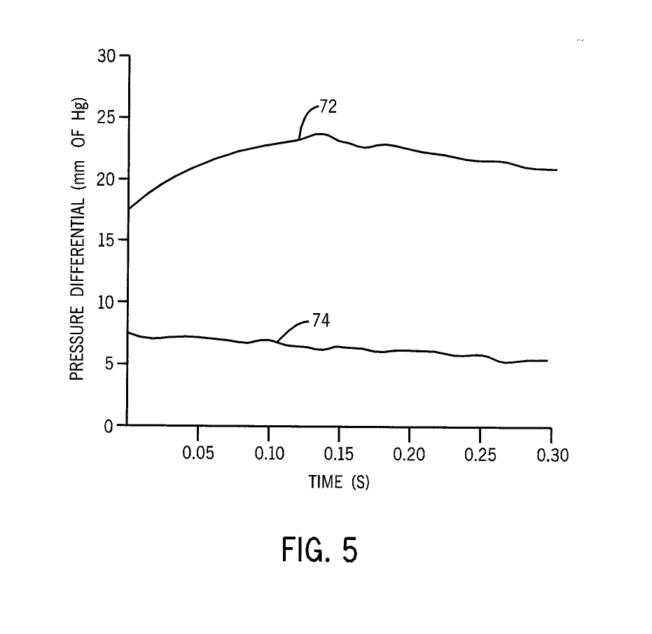

At step 112, the controller 24 analyzes pressure differential data collected as part of steps 102-110. Based on the pressure differential data collected during the initial inflation of the cuff, the controller 24 determines if the cuff 16 is the adult cuff 48 (shown in FIG. 2) or the neonatal cuff 64 (shown in FIG. 3) as described hereinafter with respect to FIG. 5. If the cuff 16 is the adult cuff 48, the controller 24 will implement the first algorithm 28 to estimate the patient's 38 blood pressure. If the cuff 16 is the neonatal cuff 64, the controller 24 will implement the second algorithm 30 to estimate the patient's 38 blood pressure. By positively identifying if the cuff 16 is the adult cuff 48 or the neonatal cuff 64, the method 100 ensures that the cuff 16 is inflated in a manner that ensures patient comfort while at the same time providing an accurate estimation of the patient's 38 blood pressure. While this embodiment is used to identify if the cuff 16 is the neonatal cuff 64 or the adult cuff 48, it should be understood that other embodiments could be used to identify additional cuff types based on the following nonlimiting list of attributes: size, brand, model, and portion of the anatomy the cuff 16 is designed to fit around.

Referring to FIG. 5, a graph representing pressure differential versus time is shown in accordance with an embodiment. Referring now to FIGS. 2, 3, and 5, an exemplary neonatal curve 72 shows the behavior of the neonatal cuff 64 and an exemplary adult curve 74 shows the behavior of the adult cuff 48. As previously described, the first and second sections of hose 60, 62 attached to the neonatal cuff 64 are generally smaller in internal diameter than the first and second sections of hose 44, 46 attached to the adult cuff 48. The generally smaller internal diameters of the first and second sections of hose 60, 62 present a greater resistance to the flow of gas, resulting in a relatively larger pressure differential during the initial inflation of the neonatal cuff 64 when compared to the pressure differential calculated during the initial inflation of the adult cuff 48. Additionally, the size and design of the cuff 48, 64 may also affect the pressure differentials measured during the initial inflation of the cuff 48, 64.

Still referring to FIG. 5, from times zero until 0.30 seconds, the pressure differential of the neonatal curve 72 ranges from 17 to 24 mm of Hg while the pressure differential of the adult curve 74 ranges from 5 to 7 mm of Hg over the same time period. By analyzing the pressure differentials collected during the initial inflation and comparing the pressure differentials to known values for a given cuff type, it is possible to identify if the cuff 16 (shown in FIG. 1) is the neonatal cuff 64 or the adult cuff 48. According to an exemplary embodiment, using a pressure differential from within the first two seconds has been observed to be well-suited to identifying cuff type. However, it should be understood by those skilled in the art that the exact period of time from which the pressure differentials are collected and the range of values used to determine the cuff type may vary depending on specifics of the NIBP system 10 (shown in FIG. 1) being used.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method for identifying a non-invasive blood pressure cuff type comprising:
inflating a cuff;
obtaining a first pressure measurement in a non-invasive blood pressure system while said inflating the cuff;
obtaining a second pressure measurement in the non-invasive blood pressure system while said inflating the cuff; and
identifying a cuff type based on the first pressure measurement and the second pressure measurement.

2. The method of claim 1, wherein said identifying the cuff type comprises calculating a difference between the first pressure measurement and the second pressure measurement.

3. The method of claim 1, wherein said identifying the cuff type comprises calculating a ratio between the first pressure measurement and the second pressure measurement.

4. The method of claim 1, wherein said obtaining the second pressure measurement comprises obtaining the second pressure measurement at generally the same time as said obtaining the first pressure measurement.

5. The method of claim 1, wherein said obtaining the first pressure measurement and said obtaining the second pressure measurement both occur within 2 seconds from said inflating the cuff.

6. The method of claim 1, wherein said obtaining the first pressure measurement comprises obtaining the first pressure measurement in a first section of hose located upstream relative to the cuff.

7. The method of claim 1, wherein said obtaining the second pressure measurement comprises obtaining the second pressure measurement in a second section of hose located downstream relative to the cuff.

8. A method for identifying a non-invasive blood pressure cuff type comprising:
inflating a cuff;
obtaining a first plurality of pressure measurements at a first location upstream relative to the cuff while said inflating the cuff;
obtaining a second plurality of pressure measurements at a second location downstream relative to the cuff while said inflating the cuff; and
identifying a cuff type based on the first plurality of pressure measurements and the second plurality of pressure measurements.

9. The method of claim 8, wherein said identifying the cuff type comprises calculating a difference based on one of the first plurality of pressure measurements and one of the second plurality of pressure measurements.

10. The method of claim 8, wherein said identifying the cuff type comprises calculating a difference based on a first average of two or more of the first plurality of pressure measurements and a second average of two or more of the second plurality of pressure measurements.

11. The method of claim 8, wherein said identifying the cuff type comprises calculating a ratio derived from one of the first plurality of pressure measurements and one of the second plurality of pressure measurements.

12. The method of claim 8, wherein said identifying the cuff type comprises calculating a ratio between a first average of two or more of the first plurality of pressure measurements and a second average of two or more of the second plurality of pressure measurements.

13. A blood pressure monitoring system comprising:
a cuff;
a first section of hose attached to the cuff;
a first transducer operatively connected to the first section of hose, wherein the first transducer is configured to obtain a first pressure measurement;
a second section of hose attached to the cuff;
a second transducer operatively connected to the second section of hose, wherein the second transducer is configured to obtain a second pressure measurement; and
a controller operatively connected to the first transducer and the second transducer, wherein the controller is configured to identify a cuff type based on the first pressure measurement and the second pressure measurement.

14. The blood pressure monitoring system of claim 13, wherein the first section of hose is located upstream relative to the cuff.

15. The blood pressure monitoring system of claim 13, wherein the second section of hose is located downstream relative to the cuff.

16. The blood pressure monitoring system of claim 13, wherein the first section of hose defines an internal diameter correlated with a specific cuff type.

17. The blood pressure monitoring system of claim 13, wherein the second section of hose defines an internal diameter correlated with a specific cuff type.

18. The blood pressure monitoring system of claim 13, wherein the controller is configured to identify the cuff type based on a difference between the first pressure measurement and the second pressure measurement.

19. The blood pressure monitoring system of claim 13, wherein the controller is configured to identify the cuff type based on a ratio between the first pressure measurement and the second pressure measurement.
